(19) United States
(12) Patent Application Publication
(10) Pub. No.: US 2026/0184493 A1
(43) Pub. Date: Jul. 2, 2026

(54) AN INDIVIDUAL DOSE POD

(71) Applicant: HALEON US HOLDINGS LLC, Wilmington, DE (US)

(72) Inventors: Gautam DEBNATH, Warren, NJ (US); Anu GADHIRAJU, Warren, NJ (US)

(21) Appl. No.: 19/131,695

(22) PCT Filed: Nov. 29, 2023
(86) PCT No.: PCT/US2023/081503

§ 371 Date: May 21, 2025

Related U.S. Application Data
(60) Provisional application No. 63/385,291, filed on Nov. 29, 2022.

(51) Int. Cl. B65D 85/804; A61J 1/14; A61K 31/09; A61K 31/137; A61K 31/138; A61K 31/167; A61K 31/485

(52) U.S. Cl. CPC B65D 85/8043; A61J 1/1431; A61K 31/09; A61K 31/137; A61K 31/138; A61K 31/167; A61K 31/485

(57) ABSTRACT

The present invention is directed to an individual dose pod for use with an automatic brewing machine. The pod includes a cup, at least one pharmaceutical active ingredient, and a lid which seals the pharmaceutical active ingredient inside the cup and provides a barrier against environmental factors.

### TECHNOLOGY FIELD

Aspects of the present invention are directed to an individual dose pod including a cup, at least one pharmaceutical active ingredient, and a lid which seals the pharmaceutical active ingredient inside the cup and provides a barrier against environmental factors.

### BACKGROUND

Single serving pods that operate with automatic brewing machines provide a hassle-free way to prepare a single serving of a beverage for consumption. This method is particularly useful for preparing beverages containing pharmaceutical active ingredients since the users of such products are typically searching for ease of use and convenience. Additionally, user-friendly, single serving pods encourage users to utilize products containing pharmaceutical active ingredients that are beneficial for their health and wellbeing by streamlining the preparation process. Brewing a single serving of a beverage also prevents waste by avoiding preparing more than needed for the user.

Single serving pods are currently available and typically designed to be used with coffee, tea, and the like. Such products are not suitable for use with pharmaceutical active ingredients due to the sensitive nature of such ingredients. In particular, standard single serving pods are not capable of protecting the sensitive pharmaceutical active ingredients from exposure to the environment. It is critical that such products be designed to maintain the quality of the ingredients by preventing exposure to water, oxygen, and other environmental factors. Therefore, sensitive pharmaceuticals must be packaged in single serving pods having barrier properties to prevent degradation and to maintain efficacy and safety for the duration of their shelf life.

Additionally, many active pharmaceutical ingredients (APIs) are required by law to be packaged in child-resistant packaging and it is vital that any single serving pods used to house such pharmaceuticals be child-resistant to prevent children from ingesting potentially dangerous substances. Current statistics show that almost a third of reported pediatric exposures to toxic substances were due to inadvertent drug ingestion, and that over seventy percent of resulting pediatric fatalities were due to unintended exposure to dangerous substances. Thus, child-resistant packaging has been an area of significant research over the past several decades.

Based on the concerns noted above, a single serving pod containing at least one pharmaceutical active ingredient, that also provides a barrier against environmental factors, is highly desirable.

### SUMMARY

Aspects of the present invention are directed to an individual dose pod for use with an automatic brewing machine comprising: a single serving pod including: a cup having a bottom portion and a side wall portion extending from the bottom portion to a rim surrounding an opening, the space between the bottom portion and the side wall portion defining an interior space, at least one pharmaceutical active ingredient provided in the interior space of the cup, and a lid affixed to the rim of the cup thereby closing the opening and securing the at least one pharmaceutical active ingredient within the interior space of the cup; wherein the lid creates a barrier being resistant to the passage of liquids and gases, and wherein the lid is a multi-layer structure including a first layer being a biaxially-oriented polyethylene terephthalate layer, a second layer being a polyethylene layer, a third layer being an aluminum foil layer, and a fourth layer being a sealing layer, such as an ionomer layer, a polyethylene layer or similar materials used alone or in combination.

The present invention further provides that the at least one pharmaceutical active ingredient is acetaminophen, phenylephrine, dextromethorphan, diphenhydramine, ibuprofen, pseudoephedrine, guaifenesin, acetylcysteine, chlorpheniramine, cetirizine, levocetirizine, doxylamine succinate, loratadine, doxylamine, or a combination thereof. In an embodiment of the present invention, the at least one pharmaceutical active ingredient is acetaminophen. In another embodiment of the present invention, the at least one pharmaceutical active ingredient is a combination of acetaminophen, phenylephrine, and dextromethorphan. In another embodiment of the present invention, the at least one pharmaceutical active ingredient is a combination of acetaminophen, phenylephrine, and diphenhydramine. In another embodiment of the present invention, the at least one pharmaceutical active ingredient is a combination of guaifenesin and dextromethorphan.

The present invention further provides that the individual dose pod is more than 70% child-resistant as determined by a Child-Resistant Test. In a preferred embodiment of the present invention, the individual dose pod is 100% child-resistant as determined by a Child-Resistant Test. Particularly, the individual dose pod is at least 70% child-resistant as determined by a Child-Resistant Test, more particularly at least 70% child-resistant or more as determine by a Child-Resistance test, such as at least 75%, at least 80%, at least 85%, at least 90%, at least 95% or 100% child-resistant as determined by a child-resistance test.

The present invention provides that the lid of the individual dose pod has appropriate moisture and/or oxygen barrier properties required for stability of a particular API (i.e., wherein the API essentially retains its physical stability and/or chemical stability and/or activity upon storage). In some embodiments, the present invention further provides that the lid of the individual dose pod has a moisture vapor transmission rate of less than 0.001 g H₂O/100 in²/24 hours. In some embodiments, the present invention further provides that the lid of the individual dose pod has an oxygen transmission rate of less than 0.005 cc/100 in²/24 hours. Particularly, there is provided an individual dose pod for use with an automatic brewing machine comprising: a single serving pod including: a cup having a bottom portion and a side wall portion extending from the bottom portion to a rim surrounding an opening, the space between the bottom portion and the side wall portion defining an interior space, at least one pharmaceutical active ingredient provided in the interior space of the cup, and a lid affixed to the rim of the cup thereby closing the opening and securing the at least one pharmaceutical active ingredient within the interior space of the cup; wherein the lid creates a barrier being resistant to the passage of liquids and/or gases, and wherein the lid is a multi-layer structure including a first layer being a biaxially-oriented polyethylene terephthalate layer, a second layer being a polyethylene layer, a third layer being an aluminum foil layer, and a fourth layer being a sealing layer. Particularly, the at least one pharmaceutical active ingredient is acetaminophen, phenylephrine, dextromethorphan, diphenhydramine, ibuprofen, pseudoephedrine, guaifenesin, acetylcysteine, chlorpheniramine, cetirizine, levocetirizine, doxylamine succinate, loratadine, doxylamine, or a combination thereof. In some embodiments the at least one pharmaceutical active ingredient is acetaminophen. In some embodiments the at least one pharmaceutical active ingredient is a combination of acetaminophen, phenylephrine, and dextromethorphan. In some embodiments the at least one pharmaceutical active ingredient is a combination of acetaminophen, phenylephrine, and diphenhydramine. In some embodiments the at least one pharmaceutical active ingredient is a combination of guaifenesin and dextromethorphan. Particularly the individual dose pod is more than 70% child-resistant as determined by a Child-Resistant Test. More particularly, the individual dose pod is 100% child-resistant as determined by a Child-Resistant Test. In some embodiments the sealing layer is an ionomer layer, a polyethylene layer, a polyethylene-based ionomer layer or combination thereof. Particularly, the lid creates a barrier being resistant to the passage of liquids and gases, particularly water and oxygen. Particularly, the lid has a moisture vapor transmission rate of less than 0.001 g H₂O/100 in²/24 hours and/or an oxygen transmission rate of less than 0.005 cc/100 in²/24 hours. In some embodiments, the lid has a moisture vapor transmission rate of less than 0.001 g H₂O/100 in²/24 hours and an oxygen transmission rate of less than 0.005 cc/100 in²/24 hours. Particularly the lid is attached to the rim of the cup by heat-sealing thereby closing the opening and securing the at least one pharmaceutical active ingredient within the interior space of the cup. In a particular embodiment there is provided an individual dose pod for use with an automatic brewing machine comprising: a single serving pod including: a cup having a bottom portion and a side wall portion extending from the bottom portion to a rim surrounding an opening, the space between the bottom portion and the side wall portion defining an interior space, at least one pharmaceutical active ingredient provided in the interior space of the cup, and a lid affixed to the rim of the cup thereby closing the opening and securing the at least one pharmaceutical active ingredient within the interior space of the cup; wherein the lid creates a barrier being resistant to the passage of liquids and/or gases, and wherein the lid is a multi-layer structure including a first layer being a biaxially-oriented polyethylene terephthalate layer, a second layer being a polyethylene layer, a third layer being an aluminum foil layer, and a fourth layer being a sealing layer, wherein the at least one pharmaceutical active ingredient is selected from the group consisting of acetaminophen, phenylephrine, dextromethorphan, diphenhydramine, ibuprofen, pseudoephedrine, guaifenesin, acetylcysteine, chlorpheniramine, cetirizine, levocetirizine, doxylamine succinate, loratadine, doxylamine, or a combination thereof, wherein the sealing layer is an ionomer layer, a polyethylene layer, a polyethylene-based ionomer layer or combination thereof; and wherein the individual dose pod is more than 70% child-resistant as determined by a Child-Resistant Test.

### BRIEF DESCRIPTION OF DRAWINGS

FIG. 1 illustrates a perspective view of a single serving pod of the present invention;

FIG. 2 illustrates an exploded view of the single serving pod of the present invention; and

FIG. 3 illustrates a cross sectional view of the layers of a lid of the present invention.

### DETAILED DESCRIPTION

Aspects of the present invention are directed to a single serving pod containing a cup, at least one pharmaceutical active ingredient, and a lid. The single serving pod may be used with an automated brewing machine to brew a single serving of a beverage containing the pharmaceutical active ingredient. The single serving pod serves as the primary packaging for the pharmaceutical active ingredient.

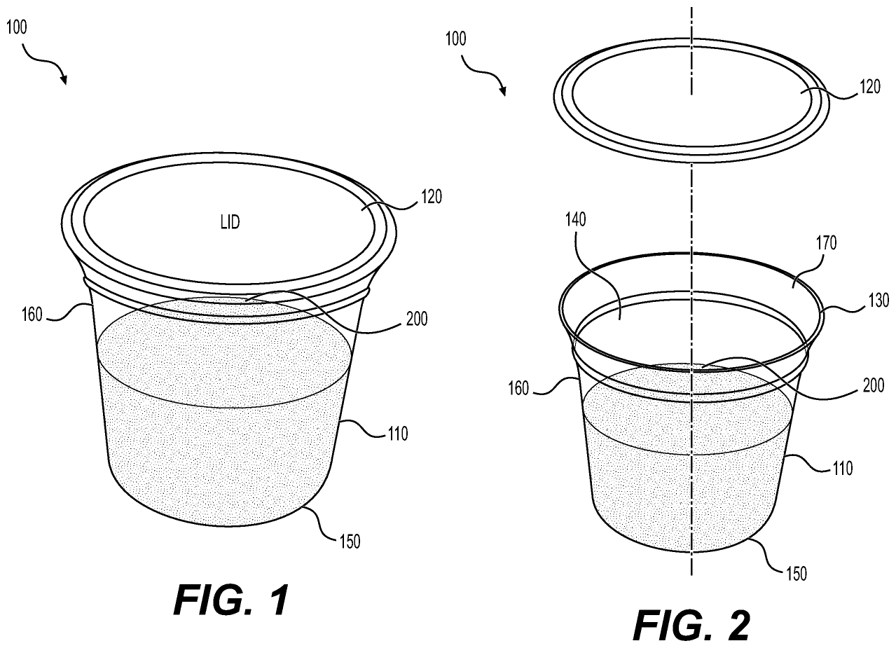

The single serving pod 100 is generally depicted in FIGS. 1 and 2. Specifically, FIG. 1 illustrates a perspective view of the single serving pod 100 and FIG. 2 illustrates an exploded view of the single serving pod 100. The single serving pod 100 includes a cup 110 and a lid 120. The cup 110 has a bottom portion 150 and a side wall portion 160 extending up from the bottom portion 150 towards a rim 130 surrounding an opening 170. The opening 170 leads to an interior space 140 defined by the side wall portion 160 and the bottom portion 150 of the cup 110. The lid 120 can be affixed onto the rim 130 of the cup 110 so as to create a permanent airtight seal that is child-resistant and has barrier resistant properties. The pharmaceutical active ingredient 200 may be stored in the interior space 140 of the cup 110 and sealed within by the lid 120. The lid 120 provides a barrier that is impermeable to the environment including, but not limited to, water and oxygen. During use, the single serving pod 100 may be placed in an automatic brewing machine to be prepared.

During the brewing cycle, the lid 120, the bottom portion 150, and/or the side wall portion 160 may be pierced or otherwise penetrated by the automatic brewing machine so that a liquid, such as water, may enter the cup 110 to be mixed with the pharmaceutical active ingredient 200 and the prepared beverage may be released from the cup 110.

Non-limiting examples of materials that may be incorporated into the laminate material of the cup 110 and/or the lid 120 include, polystyrene, ethylene vinyl alcohol, polyethylene, biaxially-oriented polyethylene terephthalate, polyethylene vinyl acetate, high-density polyethylene, cyclic olefin copolymers, biaxially oriented polyamide, ionomer, polychlorotrifluoroethylene, polypropylene, polyester, amorphous polyethylene terephthalate, polyvinyl chloride, polyethylene, aluminum foil, polyethylene terephthalate glycol, polyethylene terephthalate, orientated polyamide, low-density polyethylene, polyvinylidene chloride, or any combination thereof. The cup 110 and/or the lid 120 may be a multi-layer structure. Each layer of the laminate material of the cup 110 or the lid 120 may be attached to an adjacent layer via an adhesive.

In a preferred embodiment, the cup 110, including the bottom portion 150 and the side wall portion 160, may be a multi-layer structure made from any suitable materials that provide the desired barrier properties. In an embodiment, the cup 110 includes at least an outer polystyrene layer, a middle ethylene vinyl alcohol (EVOH) layer, and an inner polyethylene layer. In a preferred embodiment, the cup is made of an interior layer of high impact polystyrene (HIPS), a middle layer of polyvinylidene chloride, and an outer layer of polyethylene. In this embodiment, HIPS may serve as the base layer, polyvinylidene chloride may serve as the barrier layer providing barrier resistant properties to the cup in order to keep the pharmaceutical active ingredient 200 stable, and the polyethylene layer may serve as the contact and/or sealing layer. Additional adhesive layers may be provided between the layers as necessary. The multilayer structure of the cup has an overall thickness between about 500 micros and about 1500 microns. In a preferred embodiment, the multilayer structure of the cup has a thickness of about 1100 microns.

During brewing in a standard automatic brewing machine, the cup 110 may be exposed to an extremely hot liquid; thus, the cup 110 should maintain its shape and securely hold the pharmaceutical active ingredient 200 within the interior space 140 during the duration of the brewing process. Furthermore, the automatic brewing machine may pierce the bottom portion 150 of the cup 110 to allow liquid to pass through the cup 110 and into the user's container for consumption. Therefore, the structure and material of the cup provides a balance between being brittle and shatterproof i.e., able to break when subject to stress without significant deformation to allow the bottom portion 150 of the cup 110 to be easily and reliably pierced by any standard automatic brewing machine while preventing broken pieces of the cup from getting into the prepared beverage. The beverage prepared for consumption may be any volume. In certain embodiments, the prepared beverage may be about 5 fluid ounces, about 8 fluid ounces, or about 10 fluid ounces. In a preferred embodiment, the prepared beverage is about 8 fluid ounces.

The pharmaceutical active ingredient 200 provided within the interior space 140 of the cup 110 may be any compound or composition that can be used to treat and/or prevent illness and/or provide overall health and wellness benefits to the user. Non-limiting examples of a pharmaceutical active ingredient include over the counter actives, behind the counter actives, prescription actives, vitamins, minerals, plant-derived materials, energy boosting materials, probiotics, sports drinks, wellness drinks, dietary supplements, functional drinks, coffee, coffee plus milk, tea, tea plus milk, fiber, prebiotics, decongestants, anti-inflammatories, cough suppressants, expectorants, antihistamines, antitussives, pain relievers, or any combination thereof.

The pharmaceutical active ingredient 200 may be acetaminophen, phenylephrine, dextromethorphan, diphenhydramine, ibuprofen, pseudoephedrine, guaifenesin, acetylcysteine, chlorpheniramine, cetirizine, levocetirizine, doxylamine succinate, loratadine, doxylamine, or any combination thereof. The present invention may be used with any pharmaceutical active ingredient, or any combination of pharmaceutical active ingredients, or a combination of pharmaceutical active ingredients with one or more excipients. The pharmaceutical active ingredient 200 may be in any suitable form including, but not limited to, a powder, a liquid, a gel, or a tablet.

In a preferred embodiment, the pharmaceutical active ingredient may be a combination of acetaminophen, phenylephrine, and dextromethorphan. In another preferred embodiment, the pharmaceutical active ingredient may be a combination of acetaminophen, phenylephrine, and diphenhydramine. In another preferred embodiment, the pharmaceutical active ingredient may be acetaminophen.

In certain embodiments, the one or more pharmaceutical active ingredients may be present in an amount from between about 1 mg and about 2,000 mg. In embodiments wherein the pharmaceutical active ingredient 200 includes acetaminophen, it is present in an amount between about 300 mg and about 1,000 mg. In a preferred embodiment, the acetaminophen is present in an amount of about 650 mg. In another preferred embodiment, the acetaminophen is present in an amount of about 500 mg. In embodiments wherein the pharmaceutical active ingredient 200 includes phenylephrine, it is present in an amount between about 5 mg and about 30 mg. In a preferred embodiment, the phenylephrine is present in an amount of about 10 mg. In embodiments wherein the pharmaceutical active ingredient 200 includes dextromethorphan, it is present in an amount between about 5 mg and about 30 mg. In a preferred embodiment, the dextromethorphan is present in an amount of about 20 mg. In embodiments wherein the pharmaceutical active ingredient includes diphenhydramine, it is present in an amount between about 5 mg and about 50 mg. In a preferred embodiment, the diphenhydramine is present in an amount of about 25 mg.

Non-limiting example of excipients include flavors, fillers, disintegrant, glidants, lubricants, antioxidants, sweeteners, colors, preservatives, lubricants, sorbents, or any combination thereof. Suitable flavors may include, for example, mint, menthol, peppermint, wintergreen, sweet mint, spearmint, vanillin, caramel, chocolate, coffee, cinnamon, clove, citrus, lemon, green tea, white tea, chamomile, lime, orange, grape, cherry, strawberry, fruit punch, honey, honey-lemon, other fruit flavors, or any combination thereof. Suitable fillers may include, for example, maltodextrin, silicon dioxide, sodium citrate, tribasic calcium phosphate, anhydrous citric acid, sodium citrate, various grades of microcrystalline cellulose, such as Avicel PH101, Avicel PH102, & Avicel PH200, corn starch, or any combination thereof. Suitable disintegrants may include, for example, sodium starch glycolate [Explotab], crosslinked polyvinylpyrrolidone, corn starch, acacia, croscarmellose sodium [Ac-di-sol], sodium carboxymethylcellulose, veegum, alginates, or any combination thereof. Suitable glidants may include, for example, talc, corn starch, stearic acid, calcium stearate, polyethylene glycol, silicon dioxide, sodium stearyl fumarate, magnesium stearate, vegetable and mineral oils, or any combination thereof. Suitable lubricants may include, for example, magnesium stearate, stearic acid and its pharmaceutically acceptable alkali metal salts, calcium stearate, sodium stearate, Cab-O-Sil, Syloid, sodium lauryl sulfate, sodium chloride, magnesium lauryl sulfate, talc, or any combination thereof. Suitable antioxidants may include, for example, alpha tocopherol, beta tocopherol, gamma tocopherol, delta tocopherol, butylated hydroxytoluene (BHT), butylated hydroxyanisole (BHA), ascorbic acid, fumaric acid, malic acid, ascorbyl palmitate, propyl gallate, sodium ascorbate, sodium metabisulfite, or any combination thereof. Suitable sweeteners may include, for example, acesulfame potassium, aspartame, sucrose, or any combination thereof. Suitable colors may include, for example, FD&C blue no. 1, FD&C red no. 40, D&C yellow no. 10, FD&C blue no. 1, FD&C red no. 40.

In a preferred embodiment, the lid 120 may be a multi-layer structure made from any suitable materials that provide the desired barrier properties while being child-resistant. Preferably, the lid 120 includes at least a polyester layer, an adhesive layer, a primer layer, an aluminum foil layer, and a polyethylene CoEx heat seal coating layer. The lid 120 can be securely and permanently sealed onto to the rim 130 of the cup with the pharmaceutical active ingredient 200 sealed in the interior space 140 of the cup 110. The lid creates an airtight, moisture tight, barrier protective, and child-resistant seal between the lid 120 and the cup 110 that protects the pharmaceutical active ingredient 200.

The multilayer structure of the lid provides a level of moisture and child resistance that is required for pharmaceutical products. This is a higher standard than manufacturers of single serving pods used for tea, coffee, or other such beverages would require.

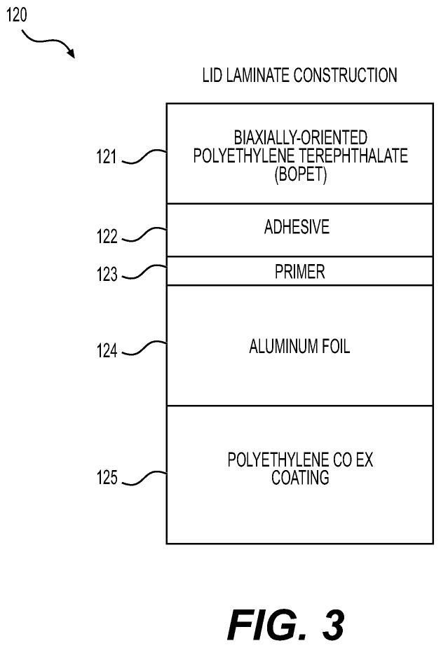

FIG. 3 illustrates a cross sectional view of the layers of the lid 120 of the present invention. The first layer is a biaxially-oriented polyethylene terephthalate (BoPET) layer. A first layer 121 is a polyester film made from polyethylene terephthalate stretched in two different directions to improve mechanical orientation. The first layer 121 provides several characteristics for the present invention, including, but not limited to, high tensile strength, durability, barrier properties, and clarity. In a preferred embodiment, the first layer 121 is clear, transparent or translucent in colour.

Furthermore, the first layer 121 of the lid 120 has a thickness between about 9 microns and about 30 microns. Preferably, the first layer 121 has a thickness of about 19.1 microns. The thickness of the first layer 121 adds to the strength of the lid 120 and further support the lid 120 being child-resistant. Typically, the thickness of a BoPET layer used for similar products would be much less; however, such similar products would not be child-resistant and thus, cannot be used with pharmaceutical ingredients.

In a preferred embodiment, the lid 120 includes a second layer 122 being an adhesive. In an embodiment, the adhesive may be an ionomer layer. The second layer 122 of the lid 120 has a thickness between about 3 micros and about 10 microns. In a preferred embodiment, the second layer 122 has a thickness of about 7.6 micros.

In a preferred embodiment, the lid 120 includes a third layer 123 being a primer. The third layer 123 of the lid 120 has a thickness between about 3 microns and 15 microns. In a preferred embodiment, the thickness of the third layer 123 is about 6.8 microns.

In a preferred embodiment, the lid 120 includes a fourth layer 124 being aluminum foil. The fourth layer 124 has a thickness between about 20 microns and about 45 microns. Preferably, the thickness of the fourth layer 124 is about 37 microns. The fourth layer 124 allows the lid 120 to be resistant to the passage of liquids and gases, including water and oxygen, among others. Specifically, the molecular structure of aluminum foil creates a strong barrier that is ideal for use with pharmaceutical ingredients due to their sensitivity to environmental factors.

The fourth layer 124, which may be referred to as a sealing layer, eliminates exposure to liquids and gases; thereby, extending the shelf life of the present invention and maintaining quality and efficacy of the pharmaceutical active ingredient 200 held within the cup 110. Furthermore, the fourth layer 124 is durable and strong while being light-weight and malleable; therefore, it is ideal for packaging products of various shapes and sizes without any loss to its barrier properties. The sealing layer may be an ionomer layer or comprised of a polymeric material selected from the group consisting of low density polyethylene (LDPE), linear low density polyethylene (LLDPE), very low density polyethylene (VLDPE), medium density polyethylene (MDPE), high density polyethylene (HDPE) and the like or blends of these polymers with or without an ionomer, and heat seal coatings. In some embodiments, the sealing layer may be an ionomer layer, a polyethylene layer, a polyethylene-based ionomer layer or a combination thereof.

In a preferred embodiment, the lid 120 includes a fifth layer 125 being a polyethylene CoEx heat seal coating layer. The fifth layer 125 has a thickness between about 15 micros and about 40 microns. In a preferred embodiment, the fifth layer 125 has a thickness of about 30 microns.

As can be seen in FIG. 3, the first layer 121 may be the outermost layer of the lid 120 on an exterior side. The second layer 122 may be provided between the first layer 121 and the third layer 123. The third layer 123 may be provided between the second layer 122 and the fourth layer 124. The fourth layer 124 may be provided between the third layer 123 and the fifth layer 125. The fifth layer 125 may be the inner most layer of the lid 120 on an interior side.

The lid 120 may have a total thickness between about 100 microns and about 150 microns. In a preferred embodiment, the lid 120 has a total thickness of about 130 microns.

The lid 120 has excellent oxygen and moisture barrier properties ideal for the pharmaceutical active ingredient 200 that may be used in the individual dose pod 100. The lid 120 may have a moisture vapor transmission rate of less than 0.001 g H₂O/100 in²/24 hours. The moisture vapor transmission rate of the lid 120 was tested at a temperature of 100° F. and a relative humidity of 90%. Furthermore, the lid 120 may have an oxygen transmission rate of less than 0.005 cc/100 in²/24 hours.

The lid 120 may be attached to the rim to seal the interior volume by any suitable method, for example, by heat-sealing, crimping, an adhesive (approved food-safe by the U.S. Food and Drug Administration (“FDA”)), etc. In one embodiment, the lid has a coating that is heat activated, causing it to attach to the rim of the base when exposed to heat. In another embodiment, the lid can be attached using ultrasonic frequency and in another embodiment the lid can be attached using induction heating. Alternatively, the lid can be attached with a contact adhesive.

Furthermore, the lid 120 provides a child-resistant and tear-resistant packaging for the single serving pod 100. Tear-resistant refers to a material that is capable of experiencing a reasonable level of stress and/or deformation without experiencing a significant loss of integrity when undergoing forces that can be applied by an individual. It is critical that the lid 120 be child-resistant so as to prevent children from getting access and potentially ingesting dangerous substances, such as the pharmaceutical active ingredient 200 that may be held within the cup 110 of the present invention.

A standardized Child-Resistant Test in accordance with 16. C.F.R. § 1700 Poison Prevention Packaging was performed on the present invention. A test failure is defined as any child who opens a package or gains access to the contents of the package. In an embodiment, the present invention can be child-resistant and can be given a rating that is referred to as the F value. The F value is the number of unit doses to which access is considered a test failure. The F value is determined based on the number of unit doses that may cause serious personal injury or serious illness to a child based on a 25-pound (11.4 kg) child. In an embodiment of the present invention, the individual dose pod 250 may be a F=1 package. In another embodiment, the individual dose pod 250 of the present invention may be a F=3 package. In another embodiment, the individual dose pod 250 of the present invention may be a F=4 package. In another embodiment, the individual dose pod 250 of the present invention may be a F=5 package. In a preferred embodiment of the present invention, the individual dose pod is a F=2 package.

The Child-Resistant Test concluded that the individual dose pod 250 is more than 70% child-resistant when the package is a F=1, F=2, F=3, F=4, or F=5 package. In another embodiment, the individual dose pod is more than 80% child-resistant when the package is a F=1, F=2, F=3, F=4, or F=5 package. In a preferred embodiment, the individual dose pod is 100% child resistant when the package is a F=1, F=2, F=3, F=4, or F=5 package. In some embodiments, the individual dose pod is an F=1 package and at least 70% or more of the children in a child-resistance test cannot access one dose. In some embodiments, the individual dose pod is an F=2 package and at least 70% or more of the children in a child-resistance test cannot access two doses or fewer. In some embodiments, the individual dose pod is an F=3 package and at least 70% or more of the children in a child-resistance test cannot access three doses or fewer. In some embodiments, the individual dose pod is an F=4 package and at least 70% or more of the children in a child-resistance test cannot access four doses or fewer. In some embodiments, the individual dose pod is an F=5 package and at least 70% or more of the children in a child-resistance test cannot access five doses or fewer. In some embodiments, the individual dose pod is an F=1 package and at least 80% or more of the children in a child-resistance test cannot access one dose. In some embodiments, the individual dose pod is an F=2 package and at least 80% or more of the children in a child-resistance test cannot access two doses or fewer. In some embodiments, the individual dose pod is an F=3 package and at least 80% or more of the children in a child-resistance test cannot access three doses or fewer. In some embodiments, the individual dose pod is an F=4 package and at least 80% or more of the children in a child-resistance test cannot access four doses or fewer. In some embodiments, the individual dose pod is an F=5 package and at least 80% or more of the children in a child-resistance test cannot access five doses or fewer. In preferred embodiments, the individual dose pod is an F=1, F=2, F=3, F=4 or F=5 package and 100% of the children in a child-resistance test cannot access one dose. For the avoidance of doubt, reference to dose or doses in this paragraph refers to dose or doses of the API contained within the pod.

Additional aspects of the present invention may be directed to methods of treating the symptoms of cold and/or flu, allergies, or other illnesses comprising preparing a beverage, as described herewith, and administering or consuming the prepared beverage. Additional aspects of the present invention may be directed to the use of a beverage prepared as described herein for the treatment of symptoms of cold and/or flu, allergies, or other illnesses.

### Example 1

Example 1 details the testing method and test result for the Child-Resistant Test conducted on the present invention to ensure its compliance with The Poison Prevention Packaging Act of 1970 Regulations and The Code of Federal Regulations Title 16: Part 1700.20. The testing method and results of the test are detailed below.

### Child-Resistant Testing Method:

The test was carried out with 14 children aged 42 months up to and including 51 months. 14% of the children were aged 42-44 months, 50% of the children were aged 45-48 months, and 0% of the children were aged 49-51 months. All of the children were suitably prepared in advance and provided with detailed information.

First, a pair of children were placed in a room, handed identical packages, and prompted by the administrator to “please try to open this for me”. Each child was then given 5 minutes to try to open his/her package. The administrator closely observed the children at all times during the test and did not influence the children in any way, either verbally or non-verbally. The children were allowed freedom of movement to work on their packages, i.e., the children were allowed to stand up, get down on the floor, bang, or pry the package. The children were also allowed to talk to each other about opening the package and could watch each other try to open the packages. If a child opened the package, the administrator said, “thank you” and took the package from the child and placed in out of the child's reach. The child was not asked to open the package a second time.

At the end of 5 minutes, the administrator demonstrated how to open the package if either child had not opened his/her package. A separate demo packaged was used by the administrator for the demonstration. Before the demonstration, the children were asked to set their packages aside and were not allowed to continue to try to open their packages. The tested asked the children to “watch me open my package” to get their attention. During the demonstration, the administrator held the package approximately two feet from the children and opened the packaged at a normal speed as if the administrator was going to use the contents. The administrator did not exaggerate any motions. The administrator also did not discuss or describe how to open the package.

After the demonstration, the administrator began a second 5-minute test period by instructing the children by a prompt of “now you try to open your packages”. If one or both children had not used their teeth to open their packages during the first 5-minute test period, the administrator instructed the children before beginning the second 5 minute test period that “you can use your teeth if you want to”. The administrator then observed the children during the second 5-minute test period or until both children opened their packages, whichever came first.

### Child-Resistant Testing Results:

Zero openings were registered in the first 5-minute test period (before demonstration). Furthermore, zero openings were registered in the second 5-minute test period (after demonstration). Zero openings were registered in the full 10-minute test period. This corresponds to a total percentage of 0.00% for the full 10-minute test, which is within the tolerance range of US 16 CFR § 1700.20. Thus, the present invention complies with the Child-Resistant Test requirements of US 16 CFR § 1700.20, as shown in Table 7.

These results are based on the assumption that an opening of the package is defined as the situation when a child gets access to 1 unit in the package. This corresponds to a failure rate of F=1.

1. An individual dose pod for use with an automatic brewing machine comprising: a single serving pod including:
a cup having a bottom portion and a side wall portion extending from the bottom portion to a rim surrounding an opening, the space between the bottom portion and the side wall portion defining an interior space,
at least one pharmaceutical active ingredient provided in the interior space of the cup, and a lid affixed to the rim of the cup thereby closing the opening and securing the at least one pharmaceutical active ingredient within the interior space of the cup;
wherein the lid creates a barrier being resistant to the passage of liquids and/or gases, and wherein the lid is a multi-layer structure including a first layer being a biaxially-oriented polyethylene terephthalate layer, a second layer being a polyethylene layer, a third layer being an aluminum foil layer, and a fourth layer being a sealing layer.

2. The individual dose pod according to claim 1, wherein the at least one pharmaceutical active ingredient is acetaminophen, phenylephrine, dextromethorphan, diphenhydramine, ibuprofen, pseudoephedrine, guaifenesin, acetylcysteine, chlorpheniramine, cetirizine, levocetirizine, doxylamine succinate, loratadine, doxylamine, or a combination thereof.

3. The individual dose pod according to claim 2, wherein the at least one pharmaceutical active ingredient is acetaminophen.

4. The individual dose pod according to claim 2, wherein the at least one pharmaceutical active ingredient is a combination of acetaminophen, phenylephrine, and dextromethorphan.

5. The individual dose pod according to claim 2, wherein the at least one pharmaceutical active ingredient is a combination of acetaminophen, phenylephrine, and diphenhydramine.

6. The individual dose pod according to claim 2, wherein the at least one pharmaceutical active ingredient is a combination of guaifenesin and dextromethorphan.

7. The individual dose pod according to claim 1, wherein the individual dose pod is more than 70% child-resistant as determined by a Child-Resistant Test.

8. The individual dose pack according to claim 7, wherein the individual dose pod is 100% child-resistant as determined by a Child-Resistant Test.

9. The individual dose pod according to claim 1, wherein the sealing layer is an ionomer layer, a polyethylene layer, a polyethylene-based ionomer layer or combination thereof.

10. The individual dose pod according to claim 1, wherein the lid has a moisture vapor transmission rate of less than 0.001 g H2O/100 in²/24 hours.

11. The individual dose pod according to claim 1, wherein the lid has an oxygen transmission rate of less than 0.005 cc/100 in²/24 hours.

12. The individual dose pod according to claim 1, wherein the lid creates a barrier being resistant to the passage of liquids and gases, particularly water and oxygen.

13. The individual dose pod according to claim 12, wherein the lid has a moisture vapor transmission rate of less than 0.001 g H2O/100 in²/24 hours and an oxygen transmission rate of less than 0.005 cc/100 in²/24 hours.

14. The individual dose pod according to claim 1, wherein the lid is attached to the rim of the cup by heat-sealing, thereby closing the opening and securing the at least one pharmaceutical active ingredient within the interior space of the cup.
